(19) United States
(12) Patent Application Publication
(10) Pub. No.: US 2020/0129105 A1
(43) Pub. Date: Apr. 30, 2020

(54) PHYSIOLOGICAL CHARACTERISTIC SENSORS AND METHODS FOR MAKING AND OPERATING SUCH SENSORS

(71) Applicant: MEDTRONIC MINIMED, INC., Northridge, CA (US)

(72) Inventors: Nicholas S. Mairs, Minneapolis, MN (US); Brian Ross, Maple Grove, MN (US)

(21) Appl. No.: 16/173,829

(22) Filed: Oct. 29, 2018

(51) Int. Cl. A61B 5/1486; A61B 5/145; A61B 5/00; A61B 5/1495; G01N 33/66

(52) U.S. Cl. CPC A61B 5/14865; A61B 5/1451; A61B 5/14532; A61B 5/7278; A61B 2562/242; A61B 5/6849; G01N 33/66; A61B 2562/125; A61B 5/1495

(57) ABSTRACT

Methods for making and operating physiological characteristic sensors are presented here. An exemplary method includes providing a quantified effect of an electrical performance parameter on a calculation of a concentration of an analyte in a fluid sample. The method includes providing a group of sensors and testing a test sensor from the group of sensors with a known concentration of the analyte in a test sample to determine the electrical performance parameter of the test sensor. Further, the method includes associating the electrical performance parameter of the test sensor with a selected sensor from the group of sensors. The method may associate the quantified effect with the selected sensor, measure an unknown concentration of the analyte with the selected sensor, and input the measured electrical performance parameter and the quantified effect into an algorithm to provide an estimated blood analyte level.

### TECHNICAL FIELD

Embodiments of the subject matter described herein relate generally to sensors for sensing and/or determining physiological characteristics of subcutaneous interstitial fluid, and more particularly, to such sensors that determine constituents of subcutaneous interstitial fluid, such as glucose levels in subcutaneous interstitial fluid, during in vivo or in vitro applications and to methods for making and operating such sensors.

### BACKGROUND

The determination of glucose levels in subcutaneous interstitial fluid is useful in a variety of applications. One particular application is for use by diabetics in combination with an insulin infusion pump system. The use of insulin pumps is frequently indicated for patients, particularly for diabetics whose conditions are best treated or stabilized by the use of insulin infusion pumps. Glucose sensors are useful in combination with such pumps, since these sensors may be used to determine glucose levels and provide information useful to the system to monitor the administration of insulin in response to actual and/or anticipated changes in blood glucose levels. For example, glucose levels are known to change in response to food and beverage intake, as well as to normal metabolic function. While certain diabetics are able to maintain proper glucose-insulin levels with conventional insulin injection or other insulin administration techniques, some individuals experience unusual problems giving rise to the need for a substantially constant glucose monitoring system to maintain an appropriate glucose-insulin balance in their bodies. Subcutaneous continuous glucose monitoring (CGM) sensors are minimally invasive portable devices able to measure (and visualize in real time) glycemia in the interstitial fluid almost continuously for approximately seven consecutive days. CGM data can be used in real time to generate alerts when glucose approaches, or exceeds, hypoglycemic or hyperglycemic thresholds.

Glucose, as a compound, is difficult to determine on a direct basis electrochemically, since its properties lead to relatively poor behavior during oxidation and/or reduction activity. Furthermore, glucose levels in subcutaneous interstitial fluid are difficult to determine inasmuch as most mechanisms for sensing and/or determining glucose levels are affected by the presence of other constituents or compounds normally found in subcutaneous interstitial fluid. For these reasons, it has been found desirable to utilize various enzymes and/or other protein materials that provide specific reactions with glucose and yield readings and/or by-products which are capable of analyses quantitatively.

For example, sensors have been outfitted with enzymes or other reagent proteins that are covalently attached to the surface of a working electrode to conduct electrochemical determinations either amperometrically or potentiometrically. When glucose and oxygen in subcutaneous interstitial fluid come into contact with the enzyme or reagent protein in the sensor, the glucose and oxygen are converted into hydrogen peroxide and gluconic acid. The hydrogen peroxide then contacts the working electrode. A voltage is applied to the working electrode, causing the hydrogen peroxide to breakdown into hydrogen, oxygen and two electrons. Generally, when glucose levels are high, more hydrogen peroxide is generated, and more electric current is generated and measured by the sensor.

Therefore, glucose sensors are highly sensitive and may behave differently according to different dimensions or properties of sensor components. As a result, performance may vary between sensors manufactured under different conditions or with different source materials, such as in different manufacturing lots, due to minute differences resulting from those manufacturing conditions or source materials. In view of these and other issues, methods for making and operating sensors designed to enhance sensing performance are desirable.

### BRIEF SUMMARY

In one aspect, the present disclosure provides a method that includes providing a quantified effect of at least one electrical performance parameter on a calculation of a concentration of an analyte in a fluid sample. The method further includes providing a group of sensors and testing a test sensor from the group of sensors with a known concentration of the analyte in a test sample to determine the at least one electrical performance parameter of the test sensor. The method also includes associating the at least one electrical performance parameter of the test sensor with a selected sensor from the group of sensors. In certain embodiments, the method may include associating the quantified effect with the selected sensor. Also, in certain embodiments, the method may include measuring an unknown concentration of the analyte in a user with the selected sensor to obtain a measured electrical performance parameter. Further, in certain embodiments, the method may include inputting the measured electrical performance parameter and the quantified effect into an algorithm to provide the user with an estimated blood analyte level, such as for example a blood glucose level.

In certain exemplary embodiments, the method may include measuring an unknown concentration of the analyte in a user with the selected sensor to obtain a measured electrical performance parameter, and inputting the measured electrical performance parameter and the quantified effect into an algorithm to predict a future concentration of the analyte in the user.

In certain exemplary embodiments of the method, the at least one electrical performance parameter is an electrical current signal (Isig), electrochemical impedance spectroscopy (EIS) output signal, and/or a counter electrode voltage (Vcntr).

In certain exemplary embodiments of the method, providing the quantified effect of the at least one electrical performance parameter on the calculation of the concentration of the analyte in the fluid sample comprises providing a transfer function equation.

In certain exemplary embodiments of the method, associating the at least one electrical performance parameter of the test sensor with the selected sensor from the group of sensors comprises printing machine readable data onto a substrate associated with the selected sensor.

In certain exemplary embodiments of the method, associating the at least one electrical performance parameter of the test sensor with the selected sensor from the group of sensors comprises printing machine readable data onto selected packaging and confining the selected sensor in the selected packaging.

In certain exemplary embodiments of the method, associating the at least one electrical performance parameter of the test sensor with the selected sensor from the group of sensors comprises associating the at least one electrical performance parameter of the test sensor with each sensor from the group of sensors.

In certain exemplary embodiments, the method further includes associating the quantified effect with the selected sensor, wherein associating the at least one electrical performance parameter of the test sensor with the selected sensor from the group of sensors and associating the quantified effect with the selected sensor comprises printing machine readable data onto a substrate associated with the selected sensor.

In certain exemplary embodiments, the method includes measuring an unknown concentration of the analyte in a user with the selected sensor to obtain a measured electrical performance parameter.

In certain exemplary embodiments, the method includes measuring an unknown concentration of the analyte in interstitial fluid in a user with the selected sensor to obtain a measured electrical performance parameter, wherein the analyte is glucose.

In another aspect, the disclosure provides a method for making a plurality of calibration-adjusted physiological characteristic sensors. The method includes providing a group of sensors and testing a test sensor from the group of sensors with a known concentration of an analyte in a test sample to determine at least one electrical performance parameter of the test sensor. Further, the method includes associating the at least one electrical performance parameter of the test sensor with remaining sensors from the group of sensors.

In certain exemplary embodiments, the method also includes enclosing each of the remaining sensors in respective packaging, wherein associating the at least one electrical performance parameter of the test sensor with the remaining sensors from the group of sensors comprises printing machine readable data onto the respective packaging.

In another exemplary embodiment, the method includes providing a quantified effect of the at least one electrical performance parameter on a calculation of a concentration of the analyte in a fluid sample, and associating the quantified effect with the remaining sensors from the group of sensors. Further, such an exemplary method may include enclosing each of the remaining sensors in respective packaging, wherein associating the at least one electrical performance parameter of the test sensor with the remaining sensors from the group of sensors and associating the quantified effect with the remaining sensors from the group of sensors comprises printing machine readable data onto the respective packaging.

In another aspect, the disclosure provides a method for operating a sensor to obtain a calibrated result adapted to assist in determining a concentration of an analyte. The method includes providing a group of sensors including the sensor and testing a test sensor from the group of sensors with a known concentration of the analyte in a test sample to determine an electrical performance parameter of the test sensor. The method also includes associating the electrical performance parameter of the test sensor with the sensor. Further, the method includes evaluating a user with the sensor to measure the electrical performance parameter to obtain a measured electrical performance parameter. Also, the method includes estimating the concentration of the analyte in the user based on the measured electrical performance parameter and a quantified effect of the electrical performance parameter on a calculation of the concentration of the analyte.

In certain embodiments of the method, estimating the concentration of the analyte in the user comprises inputting the measured electrical performance parameter and the quantified effect into an algorithm.

In another aspect, the disclosure provides a calibration-adjusted physiological characteristic sensor system including a processor for providing a quantified effect of an electrical performance parameter on a calculation of a concentration of an analyte, a sensor for measuring the concentration of the analyte, wherein the sensor is a member of a group of sensors; and a substrate associated with the sensor and including a tested electrical performance parameter in the form of machine readable data, wherein the tested electrical performance parameter is determined from testing of a test sensor from the group of sensors.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is an overhead view of an exemplary embodiment of a physiological characteristic sensor during an exemplary formation process;

FIG. 2 is a cross-sectional view taken along line 2-2 in FIG. 1 of the exemplary embodiment of a physiological characteristic sensor during formation;

FIG. 3 is a cross-sectional view of a single micro-circle in an electrode subsection in an exemplary embodiment of a physiological characteristic sensor after formation processing;

FIG. 4 is an exploded perspective view illustrating a plurality of physiological characteristic sensors formed on a substrate according to an exemplary embodiment;

FIG. 5 is a schematic view of a method for making a sensor according to an exemplary embodiment;

FIG. 6 is a schematic view of a method for using a sensor according to an exemplary embodiment;

FIG. 7 is a flow chart illustrating an exemplary method in accordance with embodiments herein; and

FIG. 8 is a flow chart illustrating an exemplary method in accordance with embodiments herein.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description. Also, while the preceding background discusses glucose sensing and exemplary physiological characteristic sensors are described as glucose sensors herein, such description is for convenience and is not limiting. The claimed subject matter may include any type of physiological characteristic sensor utilizing an embodiment of the sensor electrode described herein.

Embodiments of physiological characteristic sensors provided herein use biological elements to convert a chemical analyte in a matrix into a detectable signal. In certain embodiments, a physiological characteristic sensor of the type presented here is designed and configured for subcutaneous operation in the body of a patient. The physiological characteristic sensor includes electrodes that are electrically coupled to a suitably configured electronics module that applies the necessary excitation voltages and monitors the corresponding electrical responses (e.g., electrical current, impedance, or the like) that are indicative of physiological characteristics of the body of the patient. For the embodiment described here, the physiological characteristic sensor includes at least one working electrode, which is fabricated in a particular manner to provide the desired electrochemical characteristics. In this regard, for sensing glucose levels in a patient, the physiological characteristic sensor works according to the following chemical reactions:

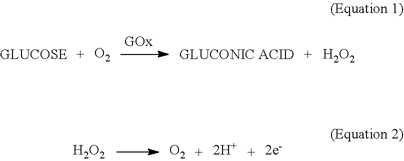

The glucose oxidase (GOx) is provided in the sensor and is encapsulated by a semipermeable membrane adjacent the working electrode. The semipermeable membrane allows for selective transport of glucose and oxygen to provide contact with the glucose oxidase. The glucose oxidase catalyzes the reaction between glucose and oxygen to yield gluconic acid and hydrogen peroxide (Equation 1). The H₂O₂ then contacts the working electrode and reacts electrochemically as shown in Equation 2 under electrocatalysis by the working electrode. The resulting current can be measured by a potentiostat. These reactions, which occur in a variety of oxidoreductases known in the art, are used in a number of sensor designs. As the size of glucose sensors and their components scale, the capability of the working electrode to efficiently electrocatalyze hydrogen peroxide is reduced. Further, minute differences between sensors caused by differences during manufacturing may result in sensor error. Embodiments herein are provided for mitigating sensor error or calibrating sensors.

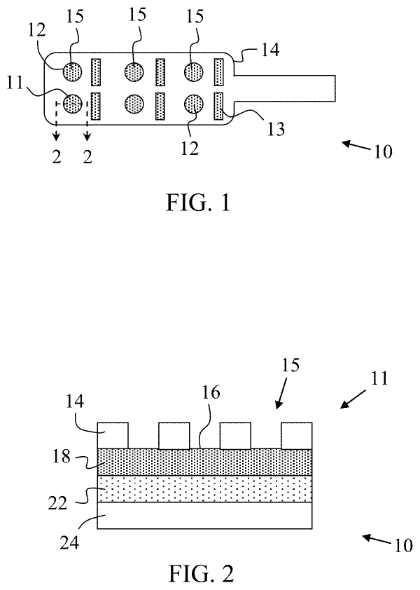

FIG. 1 is a schematic representation of an exemplary embodiment of a partially formed physiological characteristic sensor 10. FIG. 2 is a cross-sectional view of the partially formed physiological characteristic sensor 10 of FIG. 1. The sensor 10 is suitably configured to measure a physiological characteristic of the subject, e.g., a human patient. In accordance with the non-limiting embodiments presented here, the physiological characteristic of interest is glucose, and the sensor 10 generates output that is indicative of a blood glucose level of the subject. It should be appreciated that the techniques and methodologies described here may also be utilized with other sensor types if so desired.

The sensor 10 includes sensor electrodes 11 designed for subcutaneous placement at a selected site in the body of a user. When placed in this manner, the sensor electrodes 11 are exposed to the user's bodily fluids such that they can react in a detectable manner to the physiological characteristic of interest, e.g., blood glucose level. In certain embodiments, the sensor electrodes 11 may include one or more working electrodes 12, adjacent counter electrodes 13, and reference electrodes (not shown). For the embodiments described here, the sensor electrodes 11 employ thin film electrochemical sensor technology of the type used for monitoring blood glucose levels in the body. Further description of flexible thin film sensors of this general type are found in U.S. Pat. No. 5,391,250, entitled METHOD OF FABRICATING THIN FILM SENSORS, which is herein incorporated by reference. In other embodiments, different types of implantable sensor technology, such as chemical based, optical based, or the like, may be used.

The sensor electrodes 11 cooperate with sensor electronics, which may be integrated with the sensor electrodes 11 in a sensor device package, or which may be implemented in a physically distinct device or component that communicates with the sensor electrodes 11 (such as a monitor device, an infusion pump device, a controller device, or the like). In this regard, any or all of the remaining elements shown in FIG. 1 may be included in the sensor electronics, as needed to support the particular embodiment.

In the embodiment of FIG. 1, two working electrodes 12 are provided and are formed as two rows of three subsections 15. While the subsections 15 are shown as having the shape of circles, the working electrodes 12 may be formed having the shape of squares, rectangles, or other shapes as desired. While the exemplary physiological characteristic sensor 10 of FIG. 1 includes two working electrodes 12, it is envisioned that the physiological characteristic sensor 10 may include any practical number of working electrodes 12, such as one, four, six, eight, or fewer or more as desired.

In FIG. 1, each circular subsection 15 of the working electrodes 12 is formed with a surface of micro-circles having diameters of about 40 μm or about 48 μm. Other sizes may be suitable, for example, an embodiment with four working electrodes 12 may utilize circular subsections 15 formed with micro-circle having diameters of about 52 μm. As illustrated, the exemplary counter electrodes 13 are formed adjacent each circular subsection 15 of the working electrodes 12. The counter electrodes 13 are rectangular shaped, though other shapes may be utilized as desired.

The micro-circles and circular subsections 15 of the working electrodes 12 and the counter electrodes 13 defining the sensor electrodes 11 of FIG. 1 are surrounded by an electrical insulation layer 14. An exemplary insulation layer 14 is polyimide. An exemplary insulation layer has a thickness of from about 4 μm to about 10 μm, such as about 7 μm.

In FIG. 2, it can be seen that the micro-circles of the subsections 15 of the sensor electrode 11 are formed by the surfaces 16 of a metallization layer 18 that are exposed by holes, gaps, or voids formed in the overlying insulation layer 14. An exemplary metallization layer 18 is a gold material, though other suitable conductive metals may be used. The exemplary metallization layer 18 has a thickness of from about 4000 Angstroms to about 7000 Angstroms, such as about 5000 Angstroms. As shown, the exemplary metallization layer 18 is formed on an adhesion layer 22. Depending on the composition of the metallization layer 18, an adhesion layer 22 may not be needed. Specifically, certain metals do not need an adhesion layer to assist in adhesion. In an exemplary embodiment, adhesion layer 22 is a chromium-based material, though other materials suitable for assisting adhesion of the metallization layer 18 may be used. As shown, the physiological characteristic sensor 10 further includes a base layer 24. The base layer 24 may be any suitable insulator, such as, for example, polyimide. An exemplary base layer 24 has a thickness of from about 8 μm to about 18 μm, such as about 12 μm.

In an exemplary embodiment, the physiological characteristic sensor 10 is formed by sputtering the adhesion layer 22 onto the base layer 24. Then, the metallization layer 18 is sputtered onto the adhesion layer. Thereafter, the insulation layer 14 is formed on the metallization layer 18. The insulation layer 14 may be patterned after application onto the metallization layer 18 to expose the surfaces 16 of the metallization layer 18 forming the sensor electrodes 11.

After formation of the physiological characteristic sensor 10 shown in FIGS. 1 and 2, the exemplary method forms a platinum electrode deposit over the exposed surfaces 16 of the metallization layer 18. In an electrodeposition process, particles of a metal or metals are reduced from metal precursors (usually chlorides) contained in an electrolyte with acids such as sulfuric acid, nitric acid, perchloric acid, or hydrochloric acid. An electrical signal, usually with a negative potential, is applied on a conductive substrate, so that the substrate becomes negative charged (as a cathode), and a counter electrode (usually a non-polarized electrode such as a platinum electrode) becomes positive charged (as anode). Metallic ions in the solution exchange electrons with the negative substrate and are then deposited onto the substrate.

The method may include immersing the sensor electrode or electrodes 11 in a platinum electrolytic bath. An exemplary platinum electrolytic bath is a solution of hydrogen hexachloroplatinate (H₂PtCl₆) and lead acetate trihydrate (Pb(CH₃COO)₂.3H₂O), although other suitable electrolytic baths may be used.

After electrodeposition is complete, the method may include with the encapsulation of sensor layers between the electrode and a selective permeable membrane. The selective permeable membrane acts as a glucose limiting membrane during operation as a glucose sensor and limits excess glucose molecules from reacting with immobilized enzyme molecules while maximizing the availability of oxygen. In an exemplary embodiment, the sensor layers include an analyte sensing layer, such as an enzyme. An exemplary enzyme is glucose oxidase (GOx). Over the enzyme is a protein layer. An exemplary protein layer is human serum albumin (HSA). The HSA may be spray coated over the enzyme layer. An adhesion promoting composition is provided over the protein layer. The adhesion promoting composition assists in adhesion between the selective permeable membrane and the enzyme (GOx)/protein (HSA) matrix.

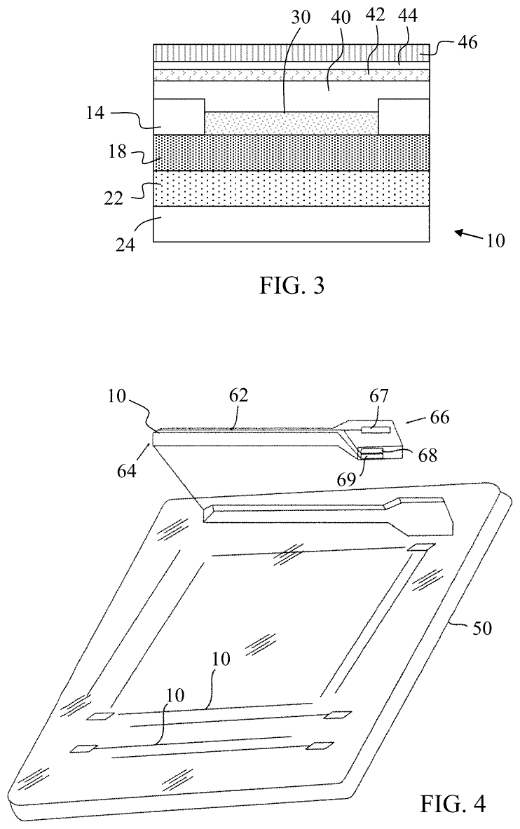

FIG. 3 further illustrates the formation of sensor layers between platinum deposit 30 and a selective permeable membrane. As shown, an analyte sensing layer 40, including a catalyst or reagent, is formed over the platinum deposit 30 (and the patterned insulation layer 14 surrounding the platinum deposit 30. An exemplary analyte sensing layer 40 includes an enzyme. An exemplary enzyme is glucose oxidase (GOx). In the illustrated embodiment, a protein layer 42 is formed over the analyte sensing layer 40. An exemplary protein layer 42 is human serum albumin (HSA). The HSA may be spray coated over the enzyme layer 40. As shown, an adhesion promoting layer 44 is provided over the protein layer. The adhesion promoting layer 44 assists in adhesion between the enzyme (GOx)/protein (HSA) layers and the selective permeable membrane 46. An exemplary selective permeable membrane 46 is a polyurethane/polyuria block copolymer composed of hexamethylene diisocyanate, aminopropyl-terminated siloxane polymer and polyethylene glycol.

While various embodiments of the sensor fabrication process have been illustrated, they are provided without limitation and other embodiments are contemplated. For example, the layout and dimensions of various components of the sensors 10 are merely for illustration and may be changed or eliminated. Further, while FIGS. 1-3 illustrate only a single sensor, it is noted that fabrication methods typically make a plurality of sensors on a single substrate, and make a plurality of substrates under the same or similar manufacturing conditions, i.e., temperature, humidity, raw and/or processed material inputs and condition, and the like.

FIG. 4 illustrates a plurality of physiological characteristic sensors 10 formed on a substrate 50 according to an exemplary embodiment. In an exemplary embodiment, the substrate 50 is a rigid flat substrate, such as a glass plate or a ceramic. Other materials that can be used for the substrate include, but are not limited to, stainless steel, aluminum, and plastic materials.

As shown, a plurality of elongated conductive traces 62 may connect the distal segment end 64 to the proximal segment end 66 of each sensor 10. At the proximal segment end 66, contact pads 67, 68, and 69 are formed.

In an exemplary embodiment, flexible sensors 10 are constructed according to so-called thin film mask techniques to include elongated thin film conductors embedded or encased between layers of a selected insulative material such as polyimide film or sheet. The sensor electrodes are exposed through one of the insulative layers for direct contact with patient fluids, such as blood and/or interstitial fluids, when the sensor is transcutaneously placed. The proximal segment 66 and the contact pads thereon are adapted for electrical connection to a suitable monitor for monitoring patient condition in response to signals derived from the sensor electrodes. The sensor electronics may be separated from the sensor by wire or be attached directly on the sensor.

After the sensor fabrication process is finished, each sensor 10 may be removed from the rigid flat substrate 50 by a suitable method, such as laser cutting. As seen in FIG. 4, the flexible sensors 10 are formed in a manner which is compatible with photolithographic mask and etch techniques, but where the sensors 10 are not physically adhered or attached directly to the substrate 50. As a result, each sensor 10 may be easily removed from substrate 50.

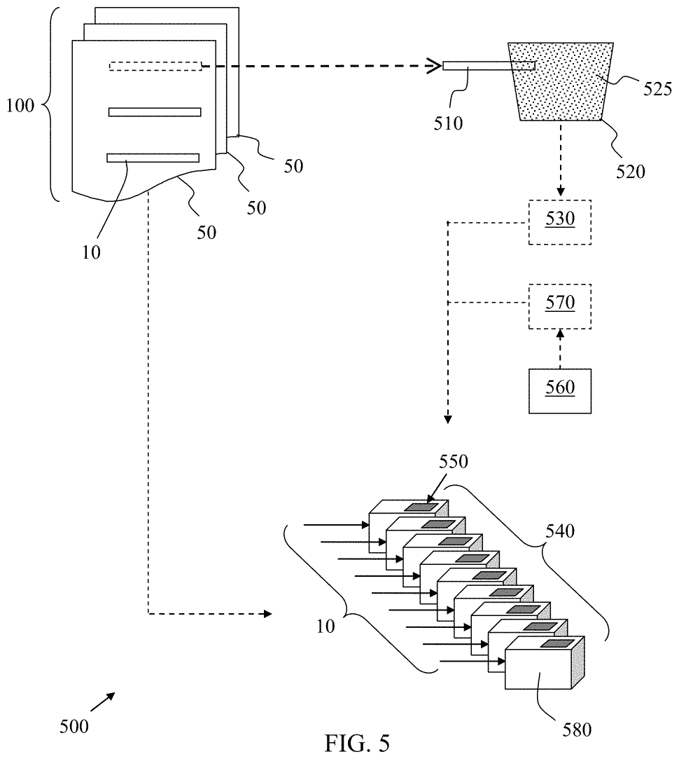

FIG. 5 is a schematic illustration of a method for making a sensor 10, as described in FIGS. 1-4. The method 500 includes providing a group 100 of sensors 10. As used herein, a group of sensors are sensors that share at least one property or characteristic such that the sensors within the group are expected to perform or behave in a substantially same way. For example, each sensor within a group may share a same source material component; or may be formed by same automated processes at a same location, such as a same manufacturing facility; or may be formed with the same critical dimensions of certain components; or may be formed during a defined time period, such as a same week or same day. In certain embodiments, a group 100 of sensors 10 may embody a single manufactured lot, i.e., sensors that are formed from the same source material components, by the same automated processes at the same location, with the same critical dimensions of certain components, and during a same defined time period.

According to the description of FIGS. 1-4, a plurality of sensors 10 may be formed on a substrate 50. Further, a plurality of substrates 50 may be processed at a same time and/or under similar manufacturing conditions, including same environment (temperature, humidity, etc.), raw and/or processed material inputs and condition. As a result, the sensors 10 formed on the substrates 50 may have the same dimensions and same component properties. Thus, the sensors 10 may be considered to form group 100. In certain embodiments, sensors 10 in a group 100 may be formed on only one substrate 50.

As shown, the method 500 includes separating a sensor 10 from a respective substrate 50 of the group 100. The sensor 10 may be considered to be a test sensor 510, i.e., a sensor to be tested. The method 500 further includes testing the test sensor 510 from the group 100 of sensors 10 with a test sample 520 having a known concentration of an analyte 525. For example, the test sample 520 may have a known concentration of glucose. Certain electrical performance parameters 530 may be determined by testing the test sample 520 with test sensor 510. For example, the electrical performance parameters 530 may include electrical current signal (Isig), an electrochemical impedance spectroscopy (EIS) output signal, and/or a counter electrode voltage (Vcntr). For example, during an EIS procedure, the EIS sensor output signal may be indicative of an impedance at a given frequency, an amplitude, and a phase angle.

As further shown in FIG. 5, the method may include associating the electrical performance parameters 530 with the sensors 10 from the group 100. For example, in FIG. 5, the method 500 prints machine readable data 550 corresponding to the electrical performance parameters 530 onto a substrate 540, such as sensor packaging. Each sensor 10 is separated from the substrates 50 and confined in, tagged by, or otherwise associated with a respective package 540.

The method 500 of FIG. 5 may further include a processor 560 for providing a quantified effect 570 of electrical performance parameter or parameters on a calculation of a concentration of an analyte in a fluid sample. Alternatively, the method may include quantifying the effect 570 of electrical performance parameter or parameters on a calculation of a concentration of an analyte in a fluid sample. For example, a study may be performed in which various electrical performance parameters are changed or held constant while concentration calculations are performed. The quantified effect 570 may be determined in the form of a transfer function equation. The quantified effect 570 may be determined by a processor and/or stored in a memory.

In certain embodiments, the machine readable data 550 may incorporate only the electrical performance parameters 530. In such embodiments, the quantified effect 570 may be applied to electrical performance parameters measured by the sensor during patient or user evaluation. In other embodiments, and as shown, the machine readable data 550 may incorporate the electrical performance parameters 530 and the quantified effect 570, or may include the output of the electrical performance parameters 530 as applied to the quantified effect 570. As shown, as a result of the method 500 of FIG. 5, a plurality of sensor products 580 are made, in which each sensor product 580 includes a sensor 10 with machine readable data 550.

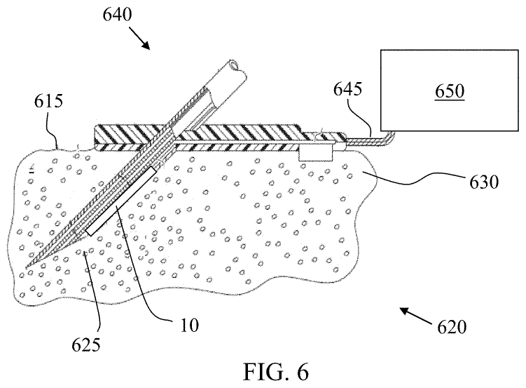

FIG. 6 illustrates a method for using a sensor 10, as fabricated according to the description of FIGS. 1-5. In FIG. 6, a sensor 10 from a sensor product 580 (shown in FIG. 5), is inserted through the skin 615 of a user 620. Specifically, the distal end of the sensor 10, including exposed electrodes, is inserted through skin 615 to a sensor placement site 625, such as into a subcutaneous tissue 625 of the user's body. Electrodes may be in contact with interstitial fluid (ISF) 630 that is usually present throughout subcutaneous tissue 625. Sensor 10 may be held in place by a sensor set 640, which may be adhesively secured to the user's skin 615. Sensor set 640 may provide for the proximal end of sensor 10 to connect to a sensor cable 645. The sensor cable 645 may further connect to a processing unit 650. The processing unit 650 may include or be coupled to a power source, such as batteries, that provides powers for sensor 10 and electrical components on a printed circuit board in processing unit 650. Electrical components of the processing unit 650 may sample a sensor signal and store sensor values in a memory.

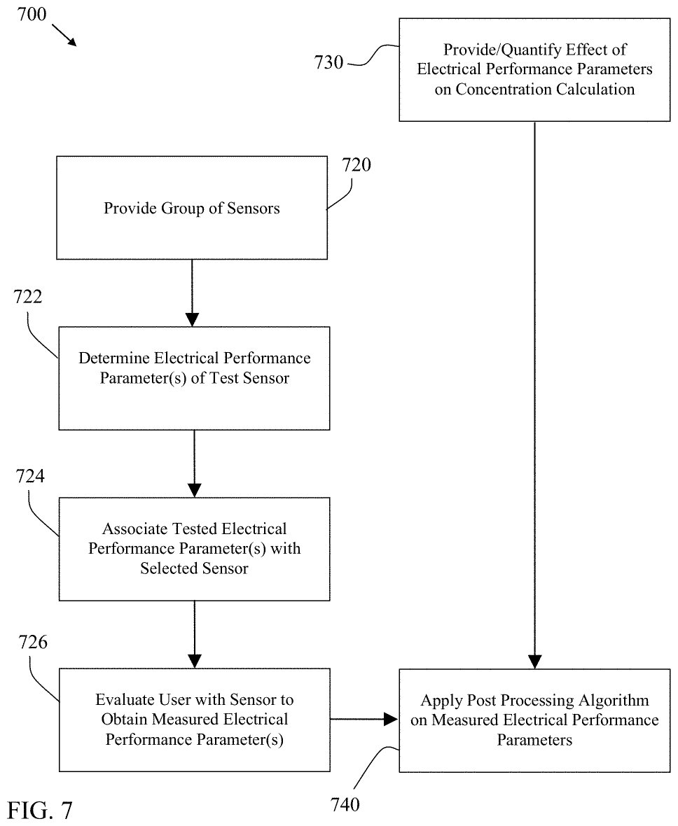

FIG. 7 provides a flow chart illustrating a method 700. Method 700 includes, at action block 720, providing a group of sensors, such as by making a group of sensors. As described above, the group of sensors may be formed on a substrate or substrates under the same conditions and with the same inputted materials.

Method 700 includes, at action block 722, testing a sensor, such as a test sensor from the group, to determine electrical parameter or parameters of the test sensor. In certain embodiments, method 700 tests the test sensor with a known concentration of an analyte, such as glucose, in a test sample to determine the electrical performance parameter or parameters of the test sensor. The electrical performance parameters may include electrical current signal (Isig), an electrochemical impedance spectroscopy (EIS) output signal, and/or a counter electrode voltage (Vcntr).

Method 700 continues with action block 724, in which the electrical performance parameter or parameters, determined in action block 722, are associated with a selected sensor from the group. For example, the electrical performance parameter or parameters may be associated with each sensor from a same manufactured substrate or from a same group as the test sensor. In certain embodiments, the electrical performance parameter(s) of the test sensor are associated with the selected sensor by printing machine readable data onto a substrate or packaging associated with the selected sensor.

Method 700 may be considered to be completed after action block 724, with the formation of a sensor for delivery to a user. Operation of the sensor may be later performed by the user. In other embodiments, method 800 continues with action block 726.

At action block 726, the selected sensor is utilized to evaluate a user to obtain measured or diagnostic electrical performance parameter(s). For example, action block 726 includes measuring an unknown concentration of the analyte in a user with the selected sensor to obtain a measured electrical performance parameter or parameters.

Parallel to action blocks 720-726, method 700 includes providing a quantified effect, or quantifying the effect, of electrical performance parameter(s) on a calculation of a concentration of the analyte in a fluid sample, at action block 730. For example, a study may be performed in which various electrical performance parameters are changed or held constant while concentration calculations are performed. The quantified effect may be determined in the form of a transfer function equation.

As further shown, method 700 includes, at action block 740, applying a post processing, i.e., post user evaluation, algorithm on the measured electrical performance parameter and the quantified effect. For example, the measured electrical performance parameter and the quantified effect may be inputted into the algorithm and the algorithm may estimate a blood analyte, e.g., blood glucose, level. The blood analyte level may be communicated to the user. In certain embodiments, the algorithm may predict a future concentration of the analyte in the user.

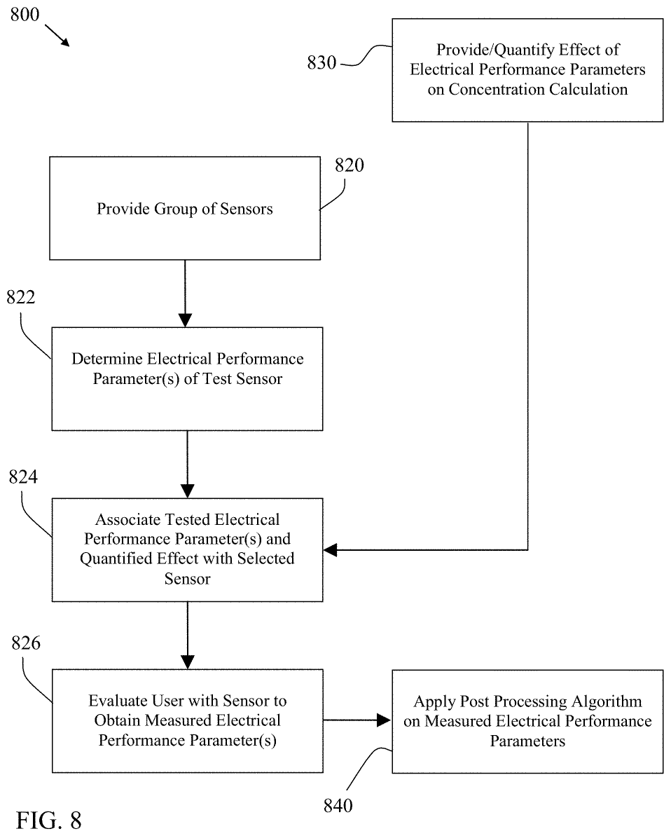

FIG. 8 provides a flow chart illustrating another method 800. Method 800 includes providing a quantified effect, or quantifying the effect, of electrical performance parameter(s) on a calculation of a concentration of the analyte in a fluid sample, at action block 830. For example, a study may be performed in which various electrical performance parameters are changed or held constant while concentration calculations are performed. The quantified effect may be determined in the form of a transfer function equation.

Method 800 further includes at action block 820 providing a group of sensors, such as by making a group of sensors. As described above, the group of sensors may be formed on a substrate or substrates under the same conditions and with the same inputted materials.

Method 800 includes, at action block 822, testing a sensor, such as a test sensor from the group, to determine electrical parameter or parameters of the test sensor. In certain embodiments, method 800 tests the test sensor with a known concentration of an analyte, such as glucose, in a test sample to determine the electrical performance parameter or parameters of the test sensor. The electrical performance parameters may include electrical current signal (Isig), an electrochemical impedance spectroscopy (EIS) output signal, and/or a counter electrode voltage (Vcntr).

Method 800 continues with action block 824, in which the electrical performance parameter or parameters, determined in action block 822, and the quantified effect of action block 830, are associated with a selected sensor from the group. For example, the electrical performance parameter or parameters may be associated with each sensor from a same manufactured substrate or from a same group as the test sensor. In certain embodiments, the electrical performance parameter(s) of the test sensor and the quantified effect are associated with the selected sensor by printing machine readable data onto a substrate or packaging associated with the selected sensor.

Method 800 may be considered to be completed after action block 824, with the formation of a sensor for delivery to a user. Operation of the sensor may be later performed by the user. In other embodiments, method 800 continues with action block 826.

At action block 826, the selected sensor is utilized to evaluate a user to obtain measured or diagnostic electrical performance parameter(s). For example, action block 826 includes measuring an unknown concentration of the analyte in a user with the selected sensor to obtain a measured electrical performance parameter or parameters.

As further shown, method 800 includes, at action block 840, applying a post processing, i.e., post user evaluation, algorithm on the measured electrical performance parameter and the quantified effect. For example, the measured electrical performance parameter and the quantified effect may be inputted into the algorithm and the algorithm may estimate a blood analyte, e.g., blood glucose, level. The blood analyte level may be communicated to the user. In certain embodiments, the algorithm may predict a future concentration of the analyte in the user.

Physiological characteristic sensors, methods for making physiological characteristic sensors designed to enhance glucose sensing performance, and methods for using physiological characteristic sensors are provided herein. As described, certain exemplary methods provide for generally quantifying the effects of electrical performance parameters, testing the electrical performance parameters of a test sensor, associating the electrical performance parameters of the test sensor to a specific sensor manufactured under same conditions, and modifying measurements of electrical performance parameter of the specific sensor made during a patient or user evaluation in view of the quantified effect and the electrical performance parameters of the test sensor.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method comprising:
providing a quantified effect of at least one electrical performance parameter on a calculation of a concentration of an analyte in a fluid sample;
providing a group of sensors;
testing a test sensor from the group of sensors with a known concentration of the analyte in a test sample to determine the at least one electrical performance parameter of the test sensor; and
associating the at least one electrical performance parameter of the test sensor with a selected sensor from the group of sensors.

2. The method of claim 1 further comprising associating the quantified effect with the selected sensor.

3. The method of claim 1 further comprising:
associating the quantified effect with the selected sensor; and
measuring an unknown concentration of the analyte in a user with the selected sensor to obtain a measured electrical performance parameter.

4. The method of claim 1 further comprising:
associating the quantified effect with the selected sensor;
measuring an unknown concentration of the analyte in a user with the selected sensor to obtain a measured electrical performance parameter; and
inputting the measured electrical performance parameter and the quantified effect into an algorithm to provide the user with an estimated blood analyte level.

5. The method of claim 1 further comprising:
measuring an unknown concentration of the analyte in a user with the selected sensor to obtain a measured electrical performance parameter; and
inputting the measured electrical performance parameter and the quantified effect into an algorithm to predict a future concentration of the analyte in the user.

6. The method of claim 1 wherein the at least one electrical performance parameter comprises an electrical current signal (Isig), an electrochemical impedance spectroscopy (EIS) output signal, and/or a counter electrode voltage (Vcntr).

7. The method of claim 1 wherein providing the quantified effect of the at least one electrical performance parameter on the calculation of the concentration of the analyte in the fluid sample comprises providing a transfer function equation.

8. The method of claim 1 wherein associating the at least one electrical performance parameter of the test sensor with the selected sensor from the group of sensors comprises printing machine readable data onto a substrate associated with the selected sensor.

9. The method of claim 1 wherein associating the at least one electrical performance parameter of the test sensor with the selected sensor from the group of sensors comprises printing machine readable data onto selected packaging and confining the selected sensor in the selected packaging.

10. The method of claim 1 wherein associating the at least one electrical performance parameter of the test sensor with the selected sensor from the group of sensors comprises associating the at least one electrical performance parameter of the test sensor with each sensor from the group of sensors.

11. The method of claim 1 further comprising associating the quantified effect with the selected sensor, wherein associating the at least one electrical performance parameter of the test sensor with the selected sensor from the group of sensors and associating the quantified effect with the selected sensor comprises printing machine readable data onto a substrate associated with the selected sensor.

12. The method of claim 1 further comprising measuring an unknown concentration of the analyte in a user with the selected sensor to obtain a measured electrical performance parameter.

13. The method of claim 1 further comprising measuring an unknown concentration of the analyte in interstitial fluid in a user with the selected sensor to obtain a measured electrical performance parameter, and wherein the analyte is glucose.

14. A method for making a plurality of calibration-adjusted physiological characteristic sensors, the method comprising:
providing a group of sensors;
testing a test sensor from the group of sensors with a known concentration of an analyte in a test sample to determine at least one electrical performance parameter of the test sensor; and
associating the at least one electrical performance parameter of the test sensor with remaining sensors from the group of sensors.

15. The method of claim 14 further comprising enclosing each of the remaining sensors in respective packaging, wherein associating the at least one electrical performance parameter of the test sensor with the remaining sensors from the group of sensors comprises printing machine readable data onto the respective packaging.

16. The method of claim 14 further comprising:
providing a quantified effect of the at least one electrical performance parameter on a calculation of a concentration of an analyte in a fluid sample; and
associating the quantified effect with the remaining sensors from the group of sensors.

17. The method of claim 14 further comprising:
providing a quantified effect of the at least one electrical performance parameter on a calculation of a concentration of an analyte in a fluid sample;
associating the quantified effect with the remaining sensors from the group of sensors; and
enclosing each of the remaining sensors in respective packaging, wherein associating the at least one electrical performance parameter of the test sensor with the remaining sensors from the group of sensors and associating the quantified effect with the remaining sensors from the group of sensors comprises printing machine readable data onto the respective packaging.

18. A method for operating a sensor to obtain a calibrated result adapted to assist in determining a concentration of an analyte, the method comprising:
providing a group of sensors including the sensor;
testing a test sensor from the group of sensors with a known concentration of the analyte in a test sample to determine an electrical performance parameter of the test sensor;
associating the electrical performance parameter of the test sensor with the sensor;
evaluating a user with the sensor to measure the electrical performance parameter to obtain a measured electrical performance parameter; and
estimating the concentration of the analyte in the user based on the measured electrical performance parameter and a quantified effect of the electrical performance parameter on a calculation of the concentration of the analyte.

19. The method of claim 18 wherein estimating the concentration of the analyte in the user comprises inputting the measured electrical performance parameter and the quantified effect into an algorithm.

20. A calibration-adjusted physiological characteristic sensor system comprising:
a processor for providing a quantified effect of an electrical performance parameter on a calculation of a concentration of an analyte;
a sensor for measuring the concentration of the analyte, wherein the sensor is a member of a group of sensors; and
a substrate associated with the sensor and including a tested electrical performance parameter in the form of machine readable data, wherein the tested electrical performance parameter is determined from testing of a test sensor from the group of sensors.
